(19) United States
(12) Patent Application Publication
(10) Pub. No.: US 2023/0201148 A1
(43) Pub. Date: Jun. 29, 2023

(54) USES OF ORNITHINE PHENYLACETATE FOR TREATING HYPERAMMONEMIA

(71) Applicant: OCERA THERAPEUTICS, INC., Hazelwood, MO (US)

(72) Inventor: Regis Vilchez, Chicago, IL (US)

(21) Appl. No.: 17/926,978

(22) PCT Filed: May 17, 2021
(86) PCT No.: PCT/US2021/032773

§ 371 Date: Nov. 21, 2022

Related U.S. Application Data
(60) Provisional application No. 63/028,874, filed on May 22, 2020.

(51) Int. Cl. A61K 31/198; A61B 5/00; A61B 5/36; A61K 45/06; A61K 9/00; A61P 1/16

(52) U.S. Cl. CPC A61K 31/198; A61B 5/4848; A61B 5/36; A61K 45/06; A61K 9/0019; A61P 1/16

(57) ABSTRACT

Embodiments of the present disclosure relate to doses of ornithine phenylacetate for treating or ameliorating hyperammonemia and the methods of administrating the same in a patient with a chronic liver disease, for example, cirrhosis. In some embodiments, the patient also has hepatic encephalopathy as a complication of the liver disease.

### BACKGROUND

Acute on-chronic liver failure is manifested initially as abnormal behavior and compromised cognition. Although the onset of hepatic encephalopathy (HE) can rarely be pinpointed clinically, it is a landmark in patients with advanced liver disease. An estimated 60% to 70% of patients with cirrhosis have at least subtle signs of neurocognitive impairment, and HE is the principal diagnosis in hospitalized patients with cirrhosis. Overt HE has a prevalence of approximately 30% in the cirrhotic population and accounts for approximately 150,000 patients being hospitalized annually in the United States. Severe HE in patients with cirrhosis is associated with a mortality of more than 50% in the first year alone.

Hepatic encephalopathy is a neuropsychiatric disorder that occurs when gut-derived toxins, primarily ammonia, bypass a failing liver, which would normally detoxify such agents; these toxins enter the circulation and cross the blood-brain barrier, resulting in impairment of neurotransmission and central nervous system function. Hepatic encephalopathy can arise in the setting of acute liver failure, chronic progressive liver disease in the context of advanced liver cirrhosis (overt HE), and/or as a result of portocaval shunting with or without liver disease. The pathogenesis of HE has been incompletely understood but the increase in venous ammonia concentrations remains central to our understanding of HE supporting the need for novel, safe, and effective venous ammonia-lowering therapies to treat as well as to prevent episodes of HE.

Dietary protein restriction had long been advocated as a strategy to indirectly reduce circulating venous ammonia in patients with cirrhosis. However, recent data have shown that this strategy is not effective in preventing HE and may harm these patients by making them more prone to muscle wasting.

Current treatment guidelines for the treatment of patients with episodic overt HE recommend administration of the non-absorbed disaccharides, lactulose, at a dose of 25 mL twice daily as first line agent, adjusted for the production of 3 bowel movements daily. Rifaximin, which alters gut microbiota, is approved for reduction in risk of overt HE recurrence.

L-ornithine L-aspartate (hereafter referred to as LOLA) is available as an IV product in Europe and Asia and may benefit patients by trapping circulating venous ammonia in the form of glutamine, although benefit in acute liver failure was not demonstrable. Phenylacetic acid and its prodrug, phenylbutyrate, have been used successfully to reduce ammonia in patients with genetic urea cycle disorders who have very high circulating glutamine levels. The approach of using only phenylacetic acid or phenylbutyrate to reduce high venous ammonia loads is not expected to work as effectively in patients with chronic liver disease because these patients typically have lower circulating glutamine levels (reduced expression of glutamine synthetase), although recent data of oral prophylaxis to prevent recurrent HE with glycerol phenylbutyrate have shown promising results. Additionally, the risk of chronic treatment and sustained glutamine depletion in cirrhotic patients with poor lean muscle mass remains a concern. As such, there remains a need for alternative and effective treatment of HE in patients with chronic liver diseases, for example, cirrhosis.

### SUMMARY

Some embodiments of the present disclosure relate to a method of treating or ameliorating hyperammonemia in a patient in need thereof, comprising: assessing or receiving information on a baseline QT interval of the patient; administering a first amount of ornithine phenylacetate to the patient during a first period of time; and administering a second amount of ornithine phenylacetate to the patient during a second period of time; wherein the first amount of ornithine phenylacetate is between about 10 g to about 30 g, and the second amount of ornithine phenylacetate is less than the first amount.

Some embodiments of the present disclosure relate to a method of treating or ameliorating hyperammonemia in a patient in need thereof, comprising: receiving or having received information on the patient's concurrent therapy with one or more QT-prolonging drugs; administering a first amount of ornithine phenylacetate to the patient during a first period of time; and administering a second amount of ornithine phenylacetate to the patient during a second period of time; wherein the first amount of ornithine phenylacetate is between about 10 g to about 30 g, and the second amount of ornithine phenylacetate is less than the first amount. In some embodiments, the methods further comprising advising the patient not to take any QT-prolonging drug concurrently or providing information regarding contraindicating concomitant use of QT-prolonging drugs to the patients.

Additional embodiments of the present disclosure relate to a method of treating or ameliorating hyperammonemia in a patient in need thereof, comprising: administering a first amount of ornithine phenylacetate to the patient during a first period of time; and administering a second amount of ornithine phenylacetate to the patient during a second period of time, wherein the first amount of ornithine phenylacetate is between about 10 g to about 30 g, and the second amount of ornithine phenylacetate is less than the first amount; and wherein the patient is not to take one or more QT-prolonging drugs concurrently. In some embodiments, the methods further comprising advising the patient not to take any QT-prolonging drug concurrently or providing information regarding contraindicating concomitant use of QT-prolonging drugs to the patients.

### DETAILED DESCRIPTION

Embodiments of the methods described herein relate to use of ornithine phenylacetate to treat or ameliorate hyperammonemia in a subject in need thereof. In some cases, the subject is experiencing or has experienced at least one overt hepatic encephalopathy episode. The methods include measuring the subject's baseline QT interval prior to the administration of ornithine phenylacetate, and may further include taking additional measurement(s) of the subject's QT interval during and after the administration of the ornithine phenylacetate to monitor the change of QT intervals. In addition, the methods may further include acquiring or receiving information on the subject's medical and prescription history including any concurrent QT-prolonging drugs. The methods may further include providing information on contraindicating concomitant use of QT-prolonging drugs to the patients. The method may also further include receiving information or assessing one or more risk factors related to QT prolongation prior to the administration of the ornithine phenylacetate. In further embodiments, the methods may include adjusting or reducing the dose of ornithine phenylacetate based on the information or assessment described herein (e.g., change of QT interval; risk factors for QT prolongation such as hypokalemia, hypomagnesemia, congenital long-QT syndrome, suspected or actual acute myocardial infarction, or concurrent therapy with one or more QT-prolonging drugs, etc.).

### Definitions

The section headings used herein are for organizational purposes only and are not to be construed as limiting the subject matter described.

Unless defined otherwise, all technical and scientific terms used herein have the same meaning as is commonly understood by one of ordinary skill in the art. The use of the term “including” as well as other forms, such as “include”, “includes,” and “included,” is not limiting. The use of the term “having” as well as other forms, such as “have”, “has,” and “had,” is not limiting. As used in this specification, whether in a transitional phrase or in the body of the claim, the terms “comprise(s)” and “comprising” are to be interpreted as having an open-ended meaning. That is, the above terms are to be interpreted synonymously with the phrases “having at least” or “including at least.” For example, when used in the context of a process, the term “comprising” means that the process includes at least the recited steps, but may include additional steps. When used in the context of a compound, composition, formulation, or device, the term “comprising” means that the compound, composition, formulation, or device includes at least the recited features or components, but may also include additional features or components.

L-Ornithine phenylacetate (also referred to as LOPA or L-OPA), a L-ornithine salt of phenylacetic acid (PAA), is a novel ammonia-lowering agent that uses pathways of ammonia removal to reduce ammonia concentration in patients with varying degrees of hepatic encephalopathy (HE).

The intravenous (IV) formulation of LOPA is a fixed-dose combination therapy that allows for alternative pathways for the excretion of ammonia in the setting of cirrhosis through the enhanced elimination of ammonia through the synergistic effects of ornithine and phenylacetic acid. Ornithine stimulates the activity of glutamine synthetase, inducing body muscle to trap circulating ammonia in the form of glutamine, which is a nontoxic carrier of ammonia. Glutamine is then conjugated with phenylacetic acid to form phenylacetylglutamine (PAGN), which is excreted in urine. This strategy prevents the eventual recirculation and degradation of glutamine by glutaminase and avoids the re-formation of ammonia.

As is well understood in the art, because of the experimental variability when X-ray diffraction patterns are measured on different instruments, the peak positions are assumed to be equal if the two theta (2θ) values agree to within 0.2° (i.e., ±0.2°). For example, the United States Pharmacopeia states that if the angular setting of the 10 strongest diffraction peaks agree to within ±0.2° with that of a reference material, and the relative intensities of the peaks do not vary by more than 20%, the identity is confirmed. Accordingly, peak positions within 0.2° of the positions recited herein are assumed to be identical.

The term “HEST” as described herein, refer to a set of criteria to assess the severity of a patient's HE. In some cases, HEST refers to Table A, B or C below. Details of HEST and uses thereof in connection with diagnosing a patient suffering from HE can be found in International Patent Appl. No. PCT/US2020/031854, which is incorporated by reference in its entirety.

“Treat,” “treatment,” or “treating,” as used herein refers to administering a pharmaceutical composition/formulation for prophylactic and/or therapeutic purposes. The term “prophylactic treatment” refers to treating a patient who is not yet suffering from a disease, but who is susceptible to, or otherwise at risk of, a particular disease, whereby the treatment reduces the likelihood that the patient will develop a disease. The term “therapeutic treatment” refers to administering treatment to a patient already suffering from a disease.

The term “asterixis” as used herein, refers to a motor disorder characterized by an inability to maintain a position due to a metabolic encephalopathy. It can be elicited on physical exam by asking patients to hold their arms straight to the front with the hands dorsiflexed as far back as possible. When asterixis is present, there will be arrhythmically occurring “flaps” of the hands downwards, followed after a brief but distinct moment by recovery back to the dorsiflexed position. In some instances, both hands will “flap” down simultaneously. In cases where patients are unable to hold their arms forward, an alternative approach to eliciting asterixis involves having the patient rest the arms prone on the bed and then asking him or her to dorsiflex the hands off the bed, again holding that position for 30 seconds. Myoclonus is distinguished by the fact that it represents not an abrupt loss of tone with a “flap” down, but rather an abrupt gain of tone with a resultant “jerk” up. Tremor is distinguished by the presence of more or less rhythmic oscillatory movement secondary to alternating contraction of agonist and antagonist musculature (Moore D P and Puri B K, Textbook of Clinical Neuropsychiatry and Behavioral Neuroscience, 2012, 3rd edition. London: Taylor & Francis Ltd.). In some embodiments of the hepatic encephalopathy staging tool (HEST) described herein, three or more flaps in thirty seconds is considered positive for asterixis.

The term “stupor” as used herein, refers to the lack of critical mental function and a level of consciousness wherein a sufferer is almost entirely unresponsive and only responds to base stimuli, such as pain. It is characterized by impairments to reactions to external stimuli. In some embodiments of the HEST described herein, a patient is considered to be in a stuporous state if the patient has severe drowsiness (can be aroused by moderate stimuli but then almost immediately drifts back to sleep), or the patient is unresponsive and the patient can be aroused only by vigorous and repeated stimuli, or the patient's speech is incomprehensible.

The term “severe drowsiness” as used herein, refers to a state in which the subject can be aroused by moderate stimuli but then almost immediately drifts back to sleep.

The term “obvious confusion” and “gross disorientation” as used herein, may include behavior such as inappropriate response to questions or commands; bewilderment; inattention to question; or combinations thereof.

As used herein, the term “coma” or “comatose” is defined as a state of unarousable unresponsiveness.

### Treatments of Hyperammonemia

Some embodiments of the present disclosure relate to a method of treating or ameliorating hyperammonemia in a patient in need thereof, comprising: assessing or receiving information on a baseline QT interval of the patient; administering a first amount of ornithine phenylacetate to the patient during a first period of time; and administering a second amount of ornithine phenylacetate to the patient during a second period of time; wherein the first amount of ornithine phenylacetate is between about 10 g to about 30 g, and the second amount of ornithine phenylacetate is less than the first amount.

In some embodiments of the method described herein, the baseline QT interval is determined as an average of two or more measurements of QT intervals prior to the administration of the first amount of ornithine phenylacetate. In one embodiment, the baseline QT interval is determined as an average of two measurements of QT interval, for example, one measurement is taken 30 minutes prior to and another measurement is taken 15 minutes prior to the administration of the first amount of ornithine phenylacetate.

In some embodiments of the method described herein, the method further comprises assessing or receiving information on a change of QT interval from the baseline QT interval during or after the administration of the first amount of ornithine phenylacetate, for example, during the first period of time. In some such embodiments, the first period of time is from about 1 hour to about 10 hours, for example, from about 2 hours to about 9 hours, from about 3 hours to about 8 hours, from about 4 hours to about 7 hours, or from about 5 hours to about 6 hours. In one embodiment, the first period of time is about 6 hours. As some non-limiting examples, the change of QT interval may be calculated as the difference between one or more QT intervals during the first period of time (e.g., a measurement taken 1, 2, 3, 4, 5 or 6 hours after the start of the first period time, or an average of any two or more measurements) and the baseline QT interval. In some embodiments, the method further comprises adjusting the first amount of ornithine phenylacetate when the change of QT interval is an increase from the baseline QT interval. In some such embodiments, the first amount of ornithine phenylacetate is about 10 g, 12 g, 14 g, 16 g, 18 g, 20 g, 22 g, 24 g, 26 g, 28 g or 30 g, or a range defined by any of the two preceding values. For example, the first amount of ornithine phenylacetate may be from about 12 g to about 28 g, from about 14 g to about 26 g, from about 16 g to about 24 g, or from about 18 g to about 22 g. In one embodiment, the first amount of ornithine phenylacetate is about 20 g. When the change of QT interval is an increase of at least 10%, 15%, 20%, 25%, 30%, 35%, 40%, 45% or 50% increase from baseline QT interval (e.g., an increase of QT interval of over 10 ms, 15 ms, 20 ms, 25 ms, 30 ms, 35 ms, 40 ms, 45 ms, 50 ms, 55 ms or 60 ms), the first amount of ornithine phenylacetate may be decreased or reduced from the standard dose described herein, for example, by about 10%, 15%, 20%, 25%, 30%, 35%, 40%, 45% or 50%.

In some embodiments, the method further comprises assessing or receiving information on a change of QT interval from the baseline QT interval during or after the administration of the second amount of ornithine phenylacetate, during the second period of time. In some embodiments, the second period of time is longer than the first period of time. In some such embodiments, the second period of time is from 12 hours to about 24 hours, or from about 16 hours to about 20 hours. In one embodiment, the second period of time is about 18 hours. In some such embodiments, a total of the first period of time and the second period of time is from about 18 hours to 36 hours, or from about 20 hours to about 30 hours. In one embodiment, the total of the first and the second period of time is about 24 hours. As some non-limiting examples, the change of QT interval may be calculated as the difference between one or more QT intervals during the second period of time (e.g., a measurement taken 1, 2, 3, 4, 5, 6, 7, 8, 9, 10, 11 or 12 hours after the start of the second period time, or an average of any two or more measurements) and the baseline QT interval. In some embodiments, the method further comprises adjusting the second amount of ornithine phenylacetate when the change of QT interval is an increase from the baseline QT interval. In some embodiments, the second amount of ornithine phenylacetate is about 5 g to about 25 g, for example, 5 g, 7.5 g, 10 g, 12.5 g, 15 g, 17.5, 20 g, 22.5 g or 25 g, or a range defined by any of the two preceding values. For example, the second amount of ornithine phenylacetate is from about 7.5 g to about 22.5 g, from about 10 g to about 20 g, or from about 12.5 g to about 17.5 g. In one embodiment, the second amount of ornithine phenylacetate is about 15 g. When the change of QT interval is an increase of at least 10%, 15%, 20%, 25%, 30%, 35%, 40%, 45% or 50% increase from baseline QT interval (e.g., an increase of QT interval of over 10 ms, 15 ms, 20 ms, 25 ms, 30 ms, 35 ms, 40 ms, 45 ms, 50 ms, 55 ms or 60 ms), the second amount of ornithine phenylacetate may be decreased or reduced from the standard dose described herein, for example, by about 10%, 15%, 20%, 25%, 30%, 35%, 40%, 45% or 50%.

In some embodiments of the method described herein, the QT interval is measured by an electrocardiogram (ECG). In some further embodiments, the QT interval is corrected for heart rate using Fridericia's formula (QTcF). In one embodiment, the baseline QTcF of the patient is less than 500 ms prior to the administration of the first amount of ornithine phenylacetate. In other words, when the baseline QTcF of the patient is equal or greater than 500 ms, such patient will be excluded from taking ornithine phenylacetate.

In some embodiments of the method described herein, the method may further comprises obtaining or having obtained information on whether the patient has one or more risk factors for QT prolongation prior to administering the first amount of ornithine phenylacetate. The risk factors for QT prolongation comprise but not limited to hypokalemia, hypomagnesemia, congenital long-QT syndrome, suspected or actual acute myocardial infarction, or concurrent therapy with one or more QT-prolonging drugs, or combinations thereof. In some such embodiments, the suspected acute myocardial infarction is determined by elevated troponin I values in conjunction with evolving or new ECG signals. A normal troponin I (TnI) level is generally between 0 and about 0.4 ng/mL. In some instances, patients having hypokalemia, hypomagnesemia, congenital long-QT syndrome, or suspected or actual acute myocardial infarction are excluded from taking ornithine phenylacetate. In some other instances, patient who is concurrently taking any QT-prolonging drug is also excluded from taking ornithine phenylacetate.

Some additional embodiments of the present disclosure relate to a method of treating or ameliorating hyperammonemia in a patient in need thereof, the method comprising: receiving or having received information on the patient's concurrent therapy with one or more QT-prolonging drugs; administering a first amount of ornithine phenylacetate to the patient during a first period of time; and administering a second amount of ornithine phenylacetate to the patient during a second period of time; wherein the first amount of ornithine phenylacetate is between about 10 g to about 30 g, and the second amount of ornithine phenylacetate is less than the first amount. Some further embodiments of the present disclosure relate to a method of treating or ameliorating hyperammonemia in a patient in need thereof, the method comprising: administering a first amount of ornithine phenylacetate to the patient during a first period of time; and administering a second amount of ornithine phenylacetate to the patient during a second period of time, wherein the first amount of ornithine phenylacetate is between about 10 g to about 30 g, and the second amount of ornithine phenylacetate is less than the first amount; and wherein the patient is not to take one or more QT-prolonging drugs concurrently.

In some embodiments, the method may further comprise assessing or receiving information on a baseline QT interval prior to the administration of the first amount of ornithine phenylacetate, and assessing or receiving information during or after the administration of the first amount of the ornithine phenylacetate (e.g., during or after the first period of time but before the start of the second period of time) to determine a change of QT interval from the baseline QT interval. In some such embodiments, the first period of time is from about 1 hour to about 10 hours, for example, from about 2 hours to about 9 hours, from about 3 hours to about 8 hours, from about 4 hours to about 7 hours, or from about 5 hours to about 6 hours. In one embodiment, the first period of time is about 6 hours. As some non-limiting examples, the change of QT interval may be calculated as the difference between one or more QT intervals during the first period of time (e.g., a measurement taken 1, 2, 3, 4, 5 or 6 hours after the start of the first period time, or an average of any two or more measurements) and the baseline QT interval. In some embodiments, the method further comprises adjusting the first amount of ornithine phenylacetate when the change of QT interval is an increase from the baseline QT interval. In some such embodiments, the first amount of ornithine phenylacetate is about 10 g, 12 g, 14 g, 16 g, 18 g, 20 g, 22 g, 24 g, 26 g, 28 g or 30 g, or a range defined by any of the two preceding values. For example, the first amount of ornithine phenylacetate may be from about 12 g to about 28 g, from about 14 g to about 26 g, from about 16 g to about 24 g, or from about 18 g to about 22 g. In one embodiment, the first amount of ornithine phenylacetate is about 20 g. When the change of QT interval is an increase of at least 10%, 15%, 20%, 25%, 30%, 35%, 40%, 45% or 50% increase from baseline QT interval (e.g., an increase of QT interval of over 10 ms, 15 ms, 20 ms, 25 ms, 30 ms, 35 ms, 40 ms, 45 ms, 50 ms, 55 ms or 60 ms), the first amount of ornithine phenylacetate may be decreased or reduced from the standard dose described herein, for example, by about 10%, 15%, 20%, 25%, 30%, 35%, 40%, 45% or 50%.

In some further embodiments, the method may further comprise assessing or receiving information on a baseline QT interval prior to the administration of the first amount of ornithine phenylacetate, and assessing or receiving information during or after the administration of the second amount of the ornithine phenylacetate (i.e., during or after the second period of time) to determine a change of QT interval from the baseline QT interval. In some such embodiments, the second period of time is from 12 hours to about 24 hours, or from about 16 hours to about 20 hours. In one embodiment, the second period of time is about 18 hours. In some such embodiments, a total of the first period of time and the second period of time is from about 18 hours to 36 hours, or from about 20 hours to about 30 hours. In one embodiment, the total of the first and the second period of time is about 24 hours. As some non-limiting examples, the change of QT interval may be calculated as the difference between one or more QT intervals during the second period of time (e.g., a measurement taken 1, 2, 3, 4, 5, 6, 7, 8, 9, 10, 11 or 12 hours after the start of the second period time, or an average of any two or more measurements) and the baseline QT interval. In some embodiments, the method further comprises adjusting the second amount of ornithine phenylacetate when the change of QT interval is an increase from the baseline QT interval. In some embodiments, the second amount of ornithine phenylacetate is about 5 g to about 25 g, for example, 5 g, 7.5 g, 10 g, 12.5 g, 15 g, 17.5, 20 g, 22.5 g or 25 g, or a range defined by any of the two preceding values. For example, the second amount of ornithine phenylacetate is from about 7.5 g to about 22.5 g, from about 10 g to about 20 g, or from about 12.5 g to about 17.5 g. In one embodiment, the second amount of ornithine phenylacetate is about 15 g. When the change of QT interval is an increase of at least 10%, 15%, 20%, 25%, 30%, 35%, 40%, 45% or 50% increase from baseline QT interval (e.g., an increase of QT interval of over 10 ms, 15 ms, 20 ms, 25 ms, 30 ms, 35 ms, 40 ms, 45 ms, 50 ms, 55 ms or 60 ms), the second amount of ornithine phenylacetate may be decreased or reduced from the standard dose described herein, for example, by about 10%, 15%, 20%, 25%, 30%, 35%, 40%, 45% or 50%.

In any embodiments of the methods described herein, the method may further comprise administering a third amount of ornithine phenylacetate following the completion of the administration of the second amount of ornithine phenylacetate during a third period of time. In some embodiments, the third amount of ornithine phenylacetate is administered immediately after the completion of the administration of the second amount of ornithine phenylacetate. In other embodiments, the third amount of ornithine phenylacetate is administered shortly after the completion of the administration of the second amount of ornithine phenylacetate (e.g., within 5 minutes to 2 hours from the completion of the administration of the second amount). In some embodiments, the third period of time is from about 2 days to about 10 days, for example from about 3 days to about 9 days, or from about 4 days to about 8 days. In one embodiment, the third period of time is 4 days (96 hours). In some embodiments, the third amount of ornithine phenylacetate is administered continuously during the third period of time. In other embodiments, the third amount of ornithine phenylacetate is administered in separate dosing periods within the third period of time. In some embodiments, the third amount of ornithine phenylacetate administered per day (24 hours) is the same or less than the second amount of ornithine phenylacetate. For example, the third amount of ornithine phenylacetate is about 5 g to about 25 g, for example, 5 g, 7.5 g, 10 g, 12.5 g, 15 g, 17.5, 20 g, 22.5 g or 25 g, or a range defined by any of the two preceding values. For example, the third amount of ornithine phenylacetate is from about 7.5 g to about 22.5 g, from about 10 g to about 20 g, or from about 12.5 g to about 17.5 g. In one embodiment, the third amount of ornithine phenylacetate is about 15 g. In some further embodiments, the method may further comprise assessing or receiving information on a baseline QT interval prior to the administration of the first amount of ornithine phenylacetate, and assessing or receiving information during the administration of the third amount of the ornithine phenylacetate (i.e., during the third period of time) to determine a change of QT interval from the baseline QT interval. In some embodiments, the method further comprises adjusting the third amount of ornithine phenylacetate when the change of QT interval is an increase from the baseline QT interval. When the change of QT interval is an increase of at least 10%, 15%, 20%, 25%, 30%, 35%, 40%, 45% or 50% increase from baseline QT interval ((e.g., an increase of QT interval of over 10 ms, 15 ms, 20 ms, 25 ms, 30 ms, 35 ms, 40 ms, 45 ms, 50 ms, 55 ms or 60 ms), the third amount of ornithine phenylacetate may be decreased or reduced from the standard dose described herein, for example, by about 10%, 15%, 20%, 25%, 30%, 35%, 40%, 45% or 50%.

In one particular example of any of the methods described herein, the first amount of ornithine phenylacetate is about 20 g and the first period of time is about 6 hours. The second amount of ornithine phenylacetate is about 15 g and the second period of time is about 18 hours. The second amount of ornithine phenylacetate is administered immediately after the completion of the administration of the first amount of ornithine phenylacetate or shortly after the completion of the administration of the first amount of ornithine phenylacetate (e.g., within 5 minutes to 1 hour from the completion of the administration of the first amount). The third amount of ornithine phenylacetate is about 15 g per day (24 hours) and the third period of time is about 4 days (96 hours). The third amount of ornithine phenylacetate is administered immediately after the completion of the administration of the second amount of ornithine phenylacetate or shortly after the completion of the administration of the second amount of ornithine phenylacetate (e.g., within 5 minutes to 1 hour from the completion of the administration of the second amount). In each period of time, ornithine phenylacetate is administered continuously. The patient's QT interval may be assessed or monitored during any of the first, second or third period of time and compared to the baseline QT interval. In addition, the first amount, second amount or third amount or ornithine phenylacetate may be adjusted or reduced if an increase of QT interval is observed (e.g., an increase of QT interval of over 10 ms, 15 ms, 20 ms, 25 ms, 30 ms, 35 ms, 40 ms, 45 ms, 50 ms, 55 ms or 60 ms). For example, the amount of ornithine phenylacetate may be reduced by 10%, 15%, 20%, 25%, 30%, 35%, 40%, 45% or 50%.

In any embodiments of the methods described herein, the first amount, second amount, or third amount of ornithine phenylacetate disclosed herein may be reduced for patients with body weight of less than 50 kg, in particular those patients who have a Child-Pugh C classification. In some such embodiments, the first amount of ornithine phenylacetate is about 15 g and the first period of time is about 6 hours. The second amount of ornithine phenylacetate is about 10 g and the second period of time is about 18 hours. The second amount of ornithine phenylacetate is administered immediately after the completion of the administration of the first amount of ornithine phenylacetate or shortly after the completion of the administration of the first amount of ornithine phenylacetate (e.g., within 5 minutes to 1 hour from the completion of the administration of the first amount). The third amount of ornithine phenylacetate is about 10 g per day (24 hours) and the third period of time is about 4 days (96 hours).

In any embodiments of the methods described herein, the first amount, second amount, or third amount of ornithine phenylacetate may be administered via different routes, for example, intravenous, oral, intraperitoneal, etc. In some embodiments, the each of the first amount, second amount and third amount of ornithine phenylacetate is administered by intravenous infusion. In one embodiment, the first amount of ornithine phenylacetate is administered by continuous intravenous infusion for 6 hours, the second amount of ornithine phenylacetate is administered by continuous intravenous infusion for 18 hours, and the third amount of ornithine phenylacetate is administered by continuous intravenous infusion for 4 days (96 hours).

In any embodiments of the method described herein, the method may further comprises advising or providing information to patient not to take any QT-prolonging drug concurrently when ornithine phenylacetate is administered or providing information regarding contraindicating concomitant use of QT-prolonging drugs to the patients. In further embodiments, the patient may also receive information regarding contraindicating concomitant use of QT-prolonging drugs prior to or after the administration of ornithine phenylacetate.

In any embodiments of the methods described herein, the QT-prolonging drugs may be selected from the group consisting of amiodarone, anagrelide, arsenic trioxide, astemizole, azithromycin, bepridil, chloroquine, chlorpromazine, cilostazol, ciprofloxacin, cisapride, citalopram, clarithromycin, cocaine, disopyramide, dofetilide, domperidone, donepezil, dronedarone, droperidol, erythromycin, escitalopram, flecainide, fluconazole, gatifloxacin, grepafloxacin, halofantrine, haloperidol, ibogaine, ibutilide, levofloxacin, levomepromazine (methotrimeprazine), levomethadyl acetate, levosulpiride, mesoridazine, methadone, moxifloxacin, ondansetron, oxaliplatin, papaverine HCl (intra-coronary), pentamidine, pimozide, probucol, procainamide, propofol, quinidine, roxithromycin, sevoflurane, sotalol, sparfloxacin, sulpiride, sultopride, terfenadine, telaprevir, terlipressin, terodiline, thioridazine, and vandetanib. In some other embodiments, the QT-prolonging drugs may be selected from the group consisting of amantadine, amisulpride, amitriptyline, amphotericin B, atazanavir, bendroflumethiazide, bendrofluazide, chloral hydrate, diphenhydramine, doxepin, esomeprazole, famotidine, fluoxetine, fluvoxamine, galantamine, garenoxacin, hydroxychloroquine, hydroxyzine, indapamide, itraconazole, ivabradine, ketoconazole, loperamide, metoclopramide, metolazone, metronidazole, nelfinavir, olanzapine, omeprazole, paroxetine, piperacillin, tazobactam, posaconazole, propafenone, quetiapine, quinine sulfate, ranolazine, sertraline, solifenacin, trazodone, torsemide (torasemide), voriconazole, and ziprasidone. In other embodiments, the QT-prolonging drugs may be selected from the group consisting of alfuzosin, apomorphine, aripiprazole, artenimol, piperaquine, asenapine, atomoxetine, bedaquiline, bendamustine, benperidol, betrixaban, bortezomib, bosutinib, buprenorphine, cabozantinib, capecitabine, ceritinib, clofazimine, clomipramine, clozapine, crizotinib, cyamemazine (cyamepromazine), dabrafenib, dasatinib, degarelix, delamanid, desipramine, deutetrabenazine, dexmedetomidine, dolasetron, efavirenz, eliglustat, epirubicin, eribulin mesylate, ezogabine (retigabine), felbamate, fingolimod, fluorouracil (5-FU), flupentixol, gemifloxacin, granisetron, hydrocodone, iloperidone, imipramine (melipramine), inotuzumab ozogamicin, isradipine, ketanserin, lapatinib, lenvatinib, lithium, lopinavir, ritonavir, melperone, midostaurin, mifepristone, mirabegron, mirtazapine, moexipril, hydrocholorothiazide, necitumumab, nicardipine, nilotinib, norfloxacin, nortriptyline, nusinersen, ofloxacin, osimertinib, oxytocin, paliperidone, palonosetron, panobinostat, pasireotide, pazopanib, perflutren lipid microspheres, perphenazine, pilsicainide, pimavanserin, pipamperone, primaquine phosphate, promethazine, prothipendyl, ribociclib, rilpivirine, risperidone, romidepsin, saquinavir, sertindole, sorafenib, sunitinib, tacrolimus, tamoxifen, telavancin, telithromycin, tetrabenazine, tiapride, tipiracil, trifluridine, tizanidine, tolterodine, toremifene, tramadol, trimipramine, tropisetron, valbenazine, vardenafil, vemurafenib, venlafaxine, vorinostat, and zotepine.

In any embodiments of the methods described herein, the patient is also receiving standard of care, for example, the patient is receiving lactulose with or without rifaximin.

In any embodiments of the methods described herein, ornithine phenylacetate is L-ornithine phenylacetate.

In any embodiments of the methods described herein, at least one of the first, second and third amount of ornithine phenylacetate is administered as an aqueous solution comprising about 100 mg/mL to about 500 mg/mL ornithine phenylacetate. In some embodiments, each of the first, second and third amount of ornithine phenylacetate is administered as an aqueous solution comprising about 200 mg/mL to about 400 mg/mL ornithine phenylacetate. In one embodiment, each of the first, second and third amount of ornithine phenylacetate is administered as an aqueous solution comprising about 300 mg/mL ornithine phenylacetate. In some such embodiment, the aqueous solution of ornithine phenylacetate has a pH of at least about 5, for example, a pH range from about 5.4 to about 6.5. In some embodiments, the aqueous solution of ornithine phenylacetate is stored at a temperature of about 2° C. to about 8° C. (e.g., 5° C.) prior to the administration.

In any embodiments of the methods described herein, the patient may have one or more conditions such as liver cirrhosis, liver decompensation, an acute liver disease, a chronic liver disease, portal hypertension, or urea cycle disorder, or a combination of the conditions. In some further embodiments, the patient may be at risk of hepatic encephalopathy (HE) or overt HE, suffering from an episode of HE or overt HE, or has suffered from one or more episodes of HE or overt HE.

In some embodiments of the methods described herein, the patient has cirrhosis. In some instances, the patient is at risk of HE or has had at least one episode of HE, for example, overt HE as a complication of cirrhosis (Stage 2, 3, or 4 as defined by Hepatic Encephalopathy Staging Tool (HEST) illustrated in Table A, B or C). In some instances, the patient may be hospitalized due to one or more hepatic encephalopathy episodes. In some such cases, the patient has received at least 4 hours to 6 hours of standard of care prior to the administration of the first amount of ornithine phenylacetate.

TABLE A

| | |
|---|---|
| Stage 0/1 | No asterixis* and no disorientation based |
| | on the following 5 questions (i.e., patient |
| | provides correct response to Questions 1, 2, 3, 4, and 5): |
| | 1. What is your name? |
| | 2. What city are we in? |
| | 3. What type of place is this? (correct answer hospital) |
| | 4. What is the year? |
| | 5. What is the month? |
| Stage 2 | Asterixis* and disorientation based on the |
| | following 5 questions, i.e., any single incorrect |
| | response qualifies the patient as Stage 2 for the following |
| | questions 1, 2, 3, 4, or 5: |
| | 1. What is your name? |
| | 2. What city are we in? |
| | 3. What type of place is this? (correct answer hospital) |
| | 4. What is the year? |
| | 5. What is the month? |
| Stage 3 | Stupor, arousable but falls asleep, responsive to verbal stimuli |
| | Obvious confusion |
| | Gross disorientation |
| Stage 4 | Coma |

*Observe for 30 seconds; 3 or more flaps in that timeframe considered positive for asterixis.

TABLE B

| | |
|---|---|
| Stage 0/1 | Patient to be considered Stage 0/1 if any of the following apply: |
| | No asterixis*/No disorientation (i.e., correct answers to all 5 questions below) |
| | No asterixis*/Yes disorientation (i.e., 1 or more incorrect answers to 5 questions |
| | below) |
| | Yes asterixis*/No disorientation (i.e., correct answers to all 5 questions below) |
| | 1. What is your name? |
| | 2. What city are we in? |
| | 3. What type of place is this? (correct answer hospital) |
| | 4. What is the year? |
| | 5. What is the month? |
| Stage 2 | Patient to be considered Stage 2 if the following applies: |
| | Yes asterixis */Yes disorientation (i.e., 1 or more incorrect answers to 5 questions |
| | below) |
| | 1. What is your name? |
| | 2. What city are we in? |
| | 3. What type of place is this? (correct answer hospital) |
| | 4. What is the year? |
| | 5. What is the month? |
| | Note: patients who are sleepy, but easily arousable and responsive to questions, can |
| | qualify as HEST Stage 2 if appropriate |
| Stage 3 | Patient to be considered Stage 3 if any of the following apply: |
| | Severe drowsiness (can be aroused by moderate stimuli but then almost |
| | immediately drifts back to sleep |
| | Stupor (unresponsiveness from which the patient can be aroused only by vigorous |
| | and repeated stimuli; incomprehensible speech) |
| | Obvious confusion/gross disorientation (inattention to questions; inappropriate |
| | response to commands or questions; bewilderment) |
| Stage 4 | Patient to be considered Stage 4 if coma is present. |
| | A coma is defined as a state of unarousable unresponsiveness. |

*Observe patient for 30 seconds; 3 or more flaps in that timeframe is considered positive for asterixis.

TABLE C

| | |
|---|---|
| Stage 0/1 | Patient to be considered Stage 0/1 if any of the following apply: |
| | No Disorientation (i.e., all correct answers for questions 1 through 7) |
| | Mild Disorientation (i.e., one incorrect answer for questions 1 through 7) |
| | 1. What is your name? |
| | 2. What year were you born? |
| | 3. What city or town do you live in? |
| | 4. What type of place is this? |
| | 5. What is the year? |
| | 6. Who is the current president (or country-specific leader)? |
| | 7. What is the month? |
| | Note: Patients who are in Stage 0/1 will be alert, responsive, and able to engage in |
| | conversation with clinical staff. |
| Stage 2 | Patient to be considered Stage 2 if the following applies: |
| | Disorientation (i.e., two or more incorrect answers for questions 1 to 7) |
| | 1. What is your name? |
| | 2. What year were you born? |
| | 3. What city or town do you live in? |
| | 4. What type of place is this? |
| | 5. What is the year? |
| | 6. Who is the current president (or country-specific leader)? |
| | 7. What is the month? |
| | Note: Patients who are in Stage 2 may be sleepy (though easily arousable) but will |
| | be responsive and able to engage in conversation with clinical staff, but may be |
| | slower to respond. |
| Stage 3 | Patient to be considered Stage 3 if any of the following apply: |
| | Gross disorientation/obvious confusion (inappropriate response to questions or |
| | commands; bewilderment; inattention to questions) |
| | Severe drowsiness (can be aroused by moderate stimuli but then almost |
| | immediately drifts back to sleep) |
| | Stupor (unresponsiveness from which the patient can be aroused only by |
| | vigorous and repeated stimuli; incomprehensible speech) |
| | Note: Patients who are in Stage 3 may have severe drowsiness where they can be |
| | aroused by moderate stimuli, but then almost immediately drift back to sleep. |
| Stage 4 | Patient to be considered Stage 4 if coma is present. |
| | A coma is defined as a state of unarousable unresponsiveness. |

It is understood by those of skill in the art that numerous and various modifications can be made without departing from the spirit of the present invention. Therefore, it should be clearly understood that the embodiments of the present disclosure are illustrative only and are not intended to limit the scope of the present invention. Any reference referred to herein is incorporated by reference for the material discussed herein, and in its entirety.

### EXAMPLE

The example below is non-limiting and is merely representative of various aspects of the present disclosure.

### Example 1

A multicenter, randomized, double-blind, placebo-controlled Phase 3 study to evaluate the efficacy, safety, and tolerability of an intravenous formulation of L-ornithine phenylacetate (LOPA) in hospitalized patients with cirrhosis and hyperammonemia associated with an episode of hepatic encephalopathy is summarized here. This study includes a substudy to evaluate the potential effect of LOPA on the QT interval corrected for heart rate using Fridericia's formula (QTcF) compared with placebo. Selected patients will undergo all of the same study procedures as patients in the main study; in addition, they will also undergo more extensive electrocardiogram (ECG) testing to quantify the effects of LOPA on the QTcF interval.

### Study Design

This is a randomized, placebo-controlled, double-blind, multicenter, and superiority Phase 3 study with 2 parallel groups. Approximately 400 patients are planned for enrollment at approximately 150 study sites around the globe. The study will enroll hospitalized patients experiencing an episode of overt HE (assessed as Stage 2, 3, or 4 per the Hepatic Encephalopathy Staging Tool [HEST]) associated with hyperammonemia as a complication of cirrhosis. Patients will be randomized to 1 of 2 treatment groups:

1. LOPA+Standard of Care (SoC).

2. Placebo+SoC.

Patients will be allocated to treatment groups in a 1:1 ratio using an interactive phone/web response system (IXRS) with randomization stratified according to 3 factors:

Rifaximin use (yes vs no).

HEST Stage 2 vs HEST Stage 3 and Stage 4.

Child-Pugh (C-P) class C vs A and B.

Lactulose±rifaximin as Standard of Care (SoC) treatment for overt HE should be administered per the clinical judgement of the investigator and usual institutional practice. Patients can be located anywhere in the hospital according to medical condition and usual clinical triage (i.e., patients need not be located in a particular unit, such as the intensive care unit to participate in this study; any hospitalized setting, including the emergency department, is acceptable).

Up to 50% of the patients will be allowed to receive rifaximin as part of the SoC. Conditions of rifaximin inclusion are as follows:
Patients who initiate or alter rifaximin treatment within 10 days of screening will be excluded.
Patients that did not receive any dose of rifaximin within 10 days prior to screening can start rifaximin as part of their treatment for HE. For these patients, rifaximin should be started at the same time as initiation of study treatment on Day 1.

The study will comprise the following study periods:

Screening/Randomization/Baseline Period. After patients diagnosed as having overt HE have received a minimum of 6 hours of SoC treatment with lactulose±rifaximin (provided dose was not initiated nor altered within 10 days prior to screening), screening activities may be initiated, starting with written informed consent. The Screening and Baseline assessments should be completed as rapidly as possible so that patients may begin receiving study treatment as soon as possible and within approximately 24 hours from the time of overt HE diagnosis. During screening, assessments and procedures will be performed and information will be collected to determine study eligibility and to establish the patient's baseline information. After completion of all screening procedures, patients will be randomized to treatment.

Treatment Period. Patients will be randomized 1:1 to receive either LOPA+SoC or placebo+SoC for up to 5 days (120 hours). The following dosing regimen is planned for this study:

First 24 hours of treatment (0 to less than 24 hours)
Loading Dose (LD): 6 hour IV infusion containing 20 g of LOPA+SoC or placebo+SoC (0 to 6 hours).
Intermediate Dose: 18 hour IV infusion containing 15 g LOPA+SoC or placebo+SoC (6 to 24 hours) immediately following the initial LD.

Remainder of treatment (not less than 24 hours to not more than 120 hours)
Maintenance Dose: for up to 4 days (from 24 hours up to 120 hours) continuous IV infusions of 15 g LOPA+SoC or placebo+SoC at a rate of 15 g per 24 hours.

The start of infusion time will be Time 0 (Day 1) and will continue through 120 hours (Day 5). The treatment period will include efficacy, safety, and PK assessments. The infusion must be stopped at least 3 hours before discharge from the hospital. Therefore, patients who are discharged from the hospital before Day 5 (120 hours) of continuous study treatment (as medically appropriate) will not receive the full 120 hours of intended treatment. End of treatment (EOT) assessments will be performed after the end of the last infusion. The last assessment should be performed within 3 hours after the end of the last infusion (±1 hour). All patients are expected to complete end-of-treatment (EOT) assessments (including those who did not receive the full 120 hours of intended treatment).

For safety, venous ammonia concentrations and PAA concentrations will be monitored while the patient is receiving study drug treatment. An independent unblinded medical monitor will review PAA and ammonia concentration data on an ongoing basis. The independent unblinded medical monitor may notify the sponsor of the need to convene an ad hoc safety review meeting with the IDSMB as outlined in the Safety Monitoring Plans.

A minimum of 216 patients with Child-Pugh Class A or B achieving clinical response by end of treatment (EOT) are required for the primary analysis to achieve 85% power; therefore, enrollment of at least 360 (180 per group) patients with Child-Pugh Class A or B is anticipated for this study. Up to an additional 40 patients with Child-Pugh Class C will also be enrolled to explore the safety and efficacy of study treatment in this patient population. While the total sample size for this study is estimated to be 400, the study may continue enrollment beyond 400 until 216 clinical events are observed in the Child-Pugh A and B population.

The duration of the study from first patient first visit to last patient last visit will be dependent on the ability of the study sites to identify and enroll eligible patients. The entire study is expected to require approximately 30 to 36 months to complete from the time the first patient is randomized.

Follow Up Period. After completion of the EOT assessments, patients will enter the Follow-Up (F/U) Period of this study for up to 30 days. The F/U Period includes safety assessments. All patients will return to the study site for two F/U Visits, which will occur on Day 14 (±2 days) and Day 30 (±2 days).

For patients who are still hospitalized for 24 hours or more post final EOI, additional safety and efficacy assessments will be performed at 2 time points: 24 hours post final EOI and at the time of pre-discharge from the hospital, as applicable.

Patients may participate in the study for a total of up to approximately 5 weeks. Patient participation in the study will be considered complete after the last follow-up assessment has been completed. The study includes a Screening/Baseline/Randomization Period of up to 24 hours, a 5-day Treatment Period (up to 120 hours or EOT), and a 30-day Follow Up (F/U) period.

Patients participating in the QTc substudy will have 12-lead ECG tracings assessed in triplicate at the time points specified in the Schedule of Study Events for the QTc Substudy (Table 1). The ECGs for the main study and the QTc substudy will be collected with a digital ECG recorder. Patients should be in the supine position with minimal procedural activity for a 10-minute window when ECG is assessed. The time points scheduled for ECGs are generally the same as those for plasma PK. In such cases, ECGs should be performed before vital sign measurements, blood draws and meals.

TABLE 1
Schedule of QTc Substudy

| | Study Period/Time Point | | | | | | | | |
|---|---|---|---|---|---|---|---|---|---|
| | Within 24 h of SOI | | | | Treatment Period Up to Day 5 (120 h) ± 2 h | | | | |
| | Day | | | | | | | | |
| | | | | | 1 | | | | |
| | 0 | | | | | | End of | | |
| Assessment/ | Screening | | 30 min | 15 min | | 3 h ± | LD 6 h ± | 9 h ± | 12 ± |
| Procedure | Assessments −22 h | Randomization −2 h | before SOI | before SOI | SOI | 30 min | 30 min | 1 h | 2 h |
| ECG [symbol] | | | X | X | | X | X | X | X |
| PK (Central | | | | | X | X | X | X | X |
| Lab)^{b} | | | | | | | | | |

| | Study Period/Time Point | | | | | | | | |
|---|---|---|---|---|---|---|---|---|---|
| | Treatment Period Up to Day 5 (120 h) ± 2 h | | | | | | Follow-Up | | |
| | Day | | | | | | | | |
| | | | | | | | 24 h post-EOI & | | |
| | | | | | | | Pre-discharge | | |
| | | | | | | | Additional F/U | | |
| | | | | | | | (only for | | |
| | 1 | 2 | 3 | 4 | 5 | | patients still | 14 | 30 |
| Assessment/ | 24 ± | 48 h ± | 72 ± | 96 ± | 120 h ± | End-of- | hospitalized >24 h | Safety F/U Visit | Safety F/U Visit |
| Procedure | 2 h | 2 h | 2 h | 2 h | 2 h | Treatment | post EOI) | (±2 days) | (±2 days) |
| ECG [symbol] | X | X | X | X | X | X | | | |
| PK (Central | X | X | X | X | X | X | | | |
| Lab)^{b} | | | | | | | | | |

ECG: electrocardiogram; D: day; EOI: end-of-infusion; EOT: end-of-treatment; F/U: follow-up; h: hour; LD: loading dose; min: minutes; PK: pharmacokinetic; SOI: start-of-infusion.
Triplicate ECGs will be assessed at Baseline (30 min, and 15 min before the SOI); 3 h ± 30 min, 6 h ± 30 min, 9 h ± 1 h, 12 h ± 2 h, 24 h ± 2h, 48 h ± 2 h, 72 h ± 2 h, 96 h ± 2 h, and 120 h ± 2 h post SOI; at 8 h (±4 h) after the final EOI (EOT visit). Baseline will be the average of the 2 pre-SOI ECG time points. ECGs should be performed before blood draws and meals. [symbol]
indicates data missing or illegible when filed

The central ECG laboratory will extract ECGs in triplicate within 1 to 2 minutes of the nominal time points for analysis. The actual time of dosing will be communicated to the central ECG laboratory. Heart rate and QT, RR, QRS, and PR intervals at Baseline and postdose will be measured in a blinded fashion. The QTc will be calculated using manual over-read QT values using the Fridericia correction method.

At each of the 2 predose time points (30 minutes and 15 minutes prior to the Start of Infusion (SIO)), a single QTcF value will be determined by averaging the triplicate ECG recordings obtained for that individual time point. The individual QTcF value at each of the 2 predose time points will be averaged, and the result will be considered the baseline QTcF. Change from baseline QTcF values (ΔQTcF) will be assessed using this baseline value.

At each of the predefined postdose time points of ECG extraction, a single QTcF value will be determined by averaging the triplicate recordings obtained from that individual time point. The individual QTcF values at each time point will be used for QTcF analysis. The QTc variables that will be determined for the QTc substudy are described below in Table 2.

TABLE 2
QTc Substudy Variables

| Variable | Description | Formula or Method |
|---|---|---|
| QTcF | Heart rate - corrected QT interval at | Fridericia's correction method |
| | each time point | |
| Baseline | Determined prior to SOI | Average of the QTcF values |
| QTcF | | determined at each of the 2 predose time |
| | | points (6 tracings total) |
| ΔQTcF | Change from Baseline in QTcF | QTcF value at specific time point |
| | | minus baseline QTcF |

QTcF: QT interval corrected for heart rate per Fridericia's method; SOI: start-of-infusion (ie, Time 0).

In addition to the standard exclusion criteria disclosed in U.S. Ser. No. 62/916,159 (which is incorporated by reference in its entirety), the following patients will be excluded from the QT substudy: 1) has a QTcF≥500 ms at Screening; 2) has a relevant risk factor for QT prolongation (e.g., hypokalemia, hypomagnesemia, congenital long-QT syndrome, concurrent therapy with QT-prolonging drugs described herein; 3) has suspected acute myocardial infarction as evidenced by serial elevated troponin I values in conjunction with evolving or new ECG signals (note: Patients with elevations in Screening troponin I should have serial troponin I levels collected every 3 hours and ECGs performed to rule out myocardial ischemia. Persistent or upward trending elevations in serial troponin I levels and evolving or new ECG changes may be indicative of myocardial ischemia and require cardiologist consult).

If a clinically significant cardiac finding is identified (including but not limited to changes from baseline in QTcF) after the start of study treatment, the investigator or a qualified designee will determine whether the patient can continue in the study and if any change in management or dose is needed.

Analysis of ECG

Heart rate, QT, and QTcF over time will be listed for each patient by treatment group. QTcF measurements will be summarized by treatment group and time point. Change from Baseline in QTcF versus time profiles will be plotted. Additionally, the following categorical analyses for the QTcF interval will be presented for each treatment group as the number and percentage of patients who meet each of the following criteria (i.e., clinical notable abnormality value) at EOT and End of Study (EOS).

Absolute QTcF interval prolongations:
Male QTcF interval>450 ms.
Female QTcF interval>480 ms.
QTcF interval>500 ms.

Change from predose Baseline in QTcF:
QTcF interval increases from Baseline>30 ms.
QTcF interval increases from Baseline>60 ms.

Change from Baseline in other ECG parameters including heart rate, PR interval, and QRS duration will also be summarized, as will new incidence of ECG morphologies.

Additional categorical analyses of PR interval and QRS interval include the number and percentage of patients who met each of the following criteria at each scheduled ECG time point:
QRS interval>120 ms and 25% increase from Baseline.
PR interval>200 ms and 25% increase from Baseline.

### Example 2

In this example, a randomized, double-blind, placebo-controlled, international study was conducted at 132 sites to evaluate ornithine phenylacetate (OP) in patients hospitalized with cirrhosis, hyperammonemia, and overt hepatic encephalopathy (OHE) stage≥2 on the Hepatic Encephalopathy Staging Tool (HEST) as shown in Table A. To qualify for study entry as stage 2 hepatic encephalopathy, both asterixis and ≥1 error in the 5 sentinel questions must have been present at screening and baseline. For recording hepatic encephalopathy response after starting study drug infusion, patients were classified as improved to stage 0/1 only if asterixis resolved and all 5 questions were answered correctly.

Eligible patients were men or nonpregnant women aged 18-75 years with cirrhosis (either evidence of or established), hospitalized with acute HE at screening (≥12 hours after HE diagnosis in the hospital, to prevent enrollment of patients who would have responded within 12 hours with SOC only [e.g., lactulose]) and at baseline (study day 1 and randomization).

Treatment (initiation of infusion) was to begin ≤1 hour (±15 minutes) after randomization. HE was assessed by HEST score, Glasgow Coma Scale, and modified-orientation log (MO-log) before start of infusion, daily during infusion (7 AM and 5 PM, ±1 hour), 3 hours after end of infusion, and on day 19 (2 weeks after end of infusion). Patients could be discharged before 5 days (120 hours) of continuous infusion, if medically appropriate. For patients remaining, HE parameters were reassessed 24 hours after end of infusion and immediately before discharge if discharge occurred during the follow-up period. All patients had a follow-up visit 2 weeks after cessation of study drug.

Continuous IV infusions of OP or placebo (5% aqueous dextrose) were given for ≤5 days (500 mL/24 hours [20.8 mL/hour] through a separate peripheral venous catheter) in addition to SOC (e.g., lactulose to achieve 2-3 bowel movements with or without rifaximin) based on investigator clinical judgment and usual institutional practice. Patients assigned to OP were randomized to 1 of 3 dosages (10, 15 or 20 g/d) according to baseline Child-Pugh score (40, 30, or 20 mg/mL for 4-6, 7-9, or 10-12 points, respectively, with each element [ascites, total bilirubin, albumin, and international normalized ratio] ranging from 1-3 points, as all patients had HE). The primary endpoint was time to confirmed clinical response using the Hepatic Encephalopathy Staging Tool in the intent-to-treat population. Safety analyses included adverse events (AEs), laboratory assessments, change from baseline in MELD score, vital signs, and electrocardiogram changes including QTcF (QT interval with Fridericia correction) and PR interval.

Safety endpoints were evaluated by treatment group and/or drug dosage. The Fisher exact test was used for between-group comparisons of treatment-emergent AEs (TEAEs) preferred terms, patients with ≥1 TEAE, treatment-emergent QTcF>30 or >60 msec, patients with QTcF>450 or >500 msec, change from baseline in PR interval (>25% increase if PR interval>200 msec), QRS complex (>25% increase if QRS complex>100 msec), and electrocardiogram heart rate (>25% decrease from baseline to <50 beats/minute, or >25% increase from baseline to >100 beats/minute).

Although not statistically significant versus placebo, it was observed that a slightly higher proportion of patients had a QTcF increase of >60 msec in the OP treated group versus placebo (11% vs 7%, respectively). An absolute QTcF of >500 msec was observed in 15% and 8% of patients who received OP and placebo, respectively.

What is claimed is:

1. A method of treating or ameliorating hyperammonemia in a patient in need thereof, comprising:
assessing or receiving information on a baseline QT interval of the patient;
administering a first amount of ornithine phenylacetate to the patient during a first period of time; and
administering a second amount of ornithine phenylacetate to the patient during a second period of time;
wherein the first amount of ornithine phenylacetate is between about 10 g to about 30 g, and the second amount of ornithine phenylacetate is less than the first amount.

2. The method of claim 1, wherein the baseline QT interval is determined as an average of two or more measurements of QT intervals prior to the administration of the first amount of ornithine phenylacetate.

3. The method of claim 3, wherein two measurements of QT interval are taken 30 minutes and 15 minutes prior to the administration of the first amount of ornithine phenylacetate.

4. The method of any one of claims 1 to 3, further comprises assessing or receiving information on a change of QT interval from the baseline QT interval during or after the administration of the first amount of ornithine phenylacetate.

5. The method of claim 4, wherein the change of QT interval is the difference between one or more QT intervals during the first period of time and the baseline QT interval.

6. The method of claim 5, further comprises adjusting the first amount of ornithine phenylacetate when the change of QT interval is an increase of more than 20% from the baseline QT interval.

7. The method of claim 4, wherein the change of QT interval is the difference between one or more QT intervals during the second period of time and the baseline QT interval.

8. The method of claim 7, further comprises adjusting the second amount of ornithine phenylacetate when the change of QT interval is an increase of more than 20% from the baseline QT interval.

9. The method of any one of claims 1 to 8, wherein the QT interval is measured by an electrocardiogram (ECG).

10. The method of any one of claims 1 to 9, wherein the QT interval is corrected for heart rate using Fridericia's formula (QTcF).

11. The method of claim 10, wherein the assessed baseline QTcF of the patient is less than 500 ms.

12. The method of any one of claims 1 to 11, further comprises obtaining or having obtained information on whether the patient has one or more risk factors for QT prolongation prior to administering the first amount of ornithine phenylacetate.

13. The method of claim 12, wherein the risk factors for QT prolongation comprise hypokalemia, hypomagnesemia, congenital long-QT syndrome, suspected or actual acute myocardial infarction, or concurrent therapy with one or more QT-prolonging drugs, or combinations thereof.

14. The method of claim 12 or 13, wherein the patient does not have hypokalemia, hypomagnesemia, or congenital long-QT syndrome.

15. The method of claim 12 or 13, wherein the patient is not being administered any QT-prolonging drug concurrently.

16. The method of claim 13, wherein the suspected acute myocardial infarction is determined by elevated troponin I values in conjunction with evolving or new ECG signals.

17. The method of claim 16, wherein the patient has a troponin I (TnI) level of between 0 and about 0.4 ng/mL.

18. A method of treating or ameliorating hyperammonemia in a patient in need thereof, the method comprising:
receiving or having received information on the patient's concurrent therapy with one or more QT-prolonging drugs;
administering a first amount of ornithine phenylacetate to the patient during a first period of time; and
administering a second amount of ornithine phenylacetate to the patient during a second period of time;
wherein the first amount of ornithine phenylacetate is between about 10 g to about 30 g, and the second amount of ornithine phenylacetate is less than the first amount.

19. A method of treating or ameliorating hyperammonemia in a patient in need thereof, the method comprising:
administering a first amount of ornithine phenylacetate to the patient during a first period of time; and
administering a second amount of ornithine phenylacetate to the patient during a second period of time, wherein the first amount of ornithine phenylacetate is between about 10 g to about 30 g, and the second amount of ornithine phenylacetate is less than the first amount; and
wherein the patient is not to take one or more QT-prolonging drugs concurrently.

20. The method of claim 13, 18 or 19, wherein the QT-prolonging drugs are selected from the group consisting of amiodarone, anagrelide, arsenic trioxide, astemizole, azithromycin, bepridil, chloroquine, chlorpromazine, cilostazol, ciprofloxacin, cisapride, citalopram, clarithromycin, cocaine, disopyramide, dofetilide, domperidone, donepezil, dronedarone, droperidol, erythromycin, escitalopram, flecainide, fluconazole, gatifloxacin, grepafloxacin, halofantrine, haloperidol, ibogaine, ibutilide, levofloxacin, levomepromazine (methotrimeprazine), levomethadyl acetate, levosulpiride, mesoridazine, methadone, moxifloxacin, ondansetron, oxaliplatin, papaverine HCl (intra-coronary), pentamidine, pimozide, probucol, procainamide, propofol, quinidine, roxithromycin, sevoflurane, sotalol, sparfloxacin, sulpiride, sultopride, terfenadine, telaprevir, terlipressin, terodiline, thioridazine, and vandetanib.

21. The method of claim 13, 18 or 19, wherein the QT-prolonging drugs are selected from the group consisting of amantadine, amisulpride, amitriptyline, amphotericin B, atazanavir, bendroflumethiazide, bendrofluazide, chloral hydrate, diphenhydramine, doxepin, esomeprazole, famotidine, fluoxetine, fluvoxamine, galantamine, garenoxacin, hydroxychloroquine, hydroxyzine, indapamide, itraconazole, ivabradine, ketoconazole, loperamide, metoclopramide, metolazone, metronidazole, nelfinavir, olanzapine, omeprazole, paroxetine, piperacillin, tazobactam, posaconazole, propafenone, quetiapine, quinine sulfate, ranolazine, sertraline, solifenacin, trazodone, torsemide (torasemide), voriconazole, and ziprasidone.

22. The method of claim 13, 18 or 19, wherein the QT-prolonging drugs are selected from the group consisting of alfuzosin, apomorphine, aripiprazole, artenimol, piperaquine, asenapine, atomoxetine, bedaquiline, bendamustine, benperidol, betrixaban, bortezomib, bosutinib, buprenorphine, cabozantinib, capecitabine, ceritinib, clofazimine, clomipramine, clozapine, crizotinib, cyamemazine (cyamepromazine), dabrafenib, dasatinib, degarelix, delamanid, desipramine, deutetrabenazine, dexmedetomidine, dolasetron, efavirenz, eliglustat, epirubicin, eribulin mesylate, ezogabine (retigabine), felbamate, fingolimod, fluorouracil (5-FU), flupentixol, gemifloxacin, granisetron, hydrocodone, iloperidone, imipramine (melipramine), inotuzumab ozogamicin, isradipine, ketanserin, lapatinib, lenvatinib, lithium, lopinavir, ritonavir, melperone, midostaurin, mifepristone, mirabegron, mirtazapine, moexipril, hydrocholorothiazide, necitumumab, nicardipine, nilotinib, norfloxacin, nortriptyline, nusinersen, ofloxacin, osimertinib, oxytocin, paliperidone, palonosetron, panobinostat, pasireotide, pazopanib, perflutren lipid microspheres, perphenazine, pilsicainide, pimavanserin, pipamperone, primaquine phosphate, promethazine, prothipendyl, ribociclib, rilpivirine, risperidone, romidepsin, saquinavir, sertindole, sorafenib, sunitinib, tacrolimus, tamoxifen, telavancin, telithromycin, tetrabenazine, tiapride, tipiracil, trifluridine, tizanidine, tolterodine, toremifene, tramadol, trimipramine, tropisetron, valbenazine, vardenafil, vemurafenib, venlafaxine, vorinostat, and zotepine.

23. The method of any one of claims 18 to 22, further comprises assessing or receiving information on a change of QT interval during the administration of the first amount or the second amount of ornithine phenylacetate, as compared from a baseline QT interval prior to the administration of the first amount of ornithine phenylacetate.

24. The method of claim 23, further comprises adjusting the first amount of ornithine phenylacetate when the change of QT interval is an increase of more than 20% from the baseline QT interval.

25. The method of claim 23, further comprises adjusting the second amount of ornithine phenylacetate when the change of QT interval is an increase of more than 20% from the baseline QT interval.

26. The method of any one of claims 1 to 25, wherein the first amount of ornithine phenylacetate is about 20 g and the first period of time is about 6 hours.

27. The method of any one of claims 1 to 26, wherein the second amount of ornithine phenylacetate is about 15 g and the second period of time is about 18 hours.

28. The method of any one of claims 1 to 27, wherein the second amount of ornithine phenylacetate is administered immediately after the completion of the administration of the first amount of ornithine phenylacetate.

29. The method of any one of claims 1 to 28, further comprises administering a third amount of ornithine phenylacetate immediately following the completion of the administration of the second amount of ornithine phenylacetate during a third period of time.

30. The method of claim 29, wherein the third period of time is about 4 days and the third amount of ornithine phenylacetate administered per day (24 hours) is the same or less than the second amount of ornithine phenylacetate.

31. The method of claim 29 or 30, further comprises assessing or receiving information on a change of QT interval from the baseline QT interval during or after the administration of the third amount of ornithine phenylacetate

32. The method of claim 31, wherein the change of QT interval is determined as the difference between one or more QT intervals during the third period of time and the baseline QT interval.

33. The method of claim 32, further comprises adjusting the third amount of ornithine phenylacetate when the change of QT interval is an increase of more than 20% from the baseline QT interval.

34. The method of any one of claims 1 to 33, wherein ornithine phenylacetate is administered via continuous intravenous infusion.

35. The method of any one of claims 1 to 34, wherein the patient is also receiving standard of care.

36. The method of claim 35, wherein the standard of care comprises lactulose with or without rifaximin.

37. The method of any one of claims 1 to 36, wherein the patient has liver cirrhosis, liver decompensation, an acute liver disease, a chronic liver disease, portal hypertension, or urea cycle disorder.

38. The method of any one of claims 1 to 37, wherein the patient is suffering from or has suffered from one or more episodes of hepatic encephalopathy.
